(19) United States
(12) Patent Application Publication
(10) Pub. No.: US 2025/0025659 A1
(43) Pub. Date: Jan. 23, 2025

(54) AUDIO SIGNAL

(71) Applicant: SynthezAl Corp., San Francisco, CA (US)

(72) Inventors: Dmytro Volodymyrovych CHERNETCHENKO, San Francisco, CA (US); Eugene Volodymyrovych NAYSHTETIK, San Francisco, CA (US); Maksym Volodymyrovych PRASOLOV, San Francisco, CA (US); Dmytro Sergiyovych LITUIEV, San Francisco, CA (US)

(73) Assignee: SynthezAl Corp., San Francisco, CA (US)

(21) Appl. No.: 18/713,461

(22) PCT Filed: Nov. 23, 2022
(86) PCT No.: PCT/GB2022/052966

§ 371 Date: May 24, 2024

(30) Foreign Application Priority Data
Nov. 24, 2021 (GB) 2116927.1

(51) Int. Cl. A61M 21/00; G10L 25/66; H04S 1/00; H04S 7/00

(52) U.S. Cl. CPC A61M 21/00; G10L 25/66; H04S 1/007; H04S 7/307; A61M 2021/0027

(57) ABSTRACT

A method of generating an output stereo signal containing binaural beats comprises: obtaining an input audio signal having a plurality of components of different frequency (S100); generating a first audio signal (S110; S120; S130) and a second audio signal based on the input audio signal, wherein components of the first audio signal have a frequency offset relative to corresponding components of the second audio signal; and forming the output stereo audio signal (S140) containing binaural beats from the first audio signal and the second audio signal.

### FIELD

The present invention relates to methods and systems for generation of audio signals for brainwave entrainment, especially audio signals that can be used in the treatment and/or alleviation of stuttering and related conditions.

### BACKGROUND

Stuttering is a speech disorder that can cause limitations in socialization, education, professional development and wellness. Adults with stuttering (AWS) may feel uncomfortable in social situations, and may develop symptoms of emotional deprivation, anxiety or depression. Two main symptoms of this disorder are the repeating of speech sounds (commonly referred to as “repetitions”) and an inability to continue speaking (commonly referred to as “blocking”). There are many other symptoms of this disorder, sharing the characteristic of preventing the stutterer from fluently speaking, as judged subjectively by the stutterer or other listeners.

Some studies directed toward reducing stuttering have shown that stuttering can be reduced by using auditory feedback. The term “auditory feedback” is used to provide the speaker (i.e., the stutterer) with his or her altered (time, frequency or phase) own speech while the speaker is speaking (usually known as altered auditory feedback methods). But such techniques have numerous limitations. In this case, a person needs to constantly interact with some kind of portable device that works on the principle of altered auditory feedback (AAF). Furthermore, the duration of action and habituation to this method is still extremely poorly understood. As a result, a person with stuttering may adapt and become desensitized to the device after constant use, resulting in a decrease in the effectiveness of the technique.

Many scientific studies have demonstrated the ability of auditory stimulation to help premature infants gain weight, autistic children communicate, and stroke patients regain speech and mobility. Auditory stimulation has also been shown to be beneficial in controlling chronic pain and as an effective method to reduce anxiety and depression. In addition, there is evidence that auditory stimulation stimulates memories and assists in restoring cognitive function in patients suffering from Alzheimer's disease.

### SUMMARY

According to an aspect of the invention, there is provided a method of generating an output stereo signal containing binaural beats comprising: obtaining an input audio signal having a plurality of components of different frequency; generating a first audio signal and a second audio signal based on the input audio signal, wherein components of the first audio signal have a frequency offset relative to corresponding components of the second audio signal; and forming the output stereo audio signal containing binaural beats from the first audio signal and the second audio signal.

According to an aspect of the invention, there is provided a system for generating a stereo output audio signal containing binaural beats comprising: an audio signal input unit, configured to obtain an input audio signal having a plurality of components of different frequency; a processing unit configured to generate a first audio signal and a second audio signal based on the input audio signal, wherein components of the first audio signal have a frequency offset relative to corresponding components of the second audio signal; and a stereo output audio signal generating unit, configured to form the stereo output audio signal containing binaural beats from the first audio signal and the second audio signal.

According to an aspect of the invention, there is provided a method of treatment for stuttering in an individual, comprising playing a stereo audio signal containing binaural beats to an individual.

According to an aspect of the invention, there is provided a stereo audio signal containing binaural beats for use in a method of treatment of stuttering.

According to an aspect of the invention, there is provided a computer program comprising code means that, when executed by a computer system, perform the above-mentioned method of generating an output stereo signal containing binaural beats.

According to an aspect of the invention, there is provided a non-transitory storage medium storing the stereo output audio signal generated according to the above-mentioned method of generating an output stereo signal containing binaural beats or using the system for generating a stereo output audio signal containing binaural beats.

### BRIEF DESCRIPTION OF THE DRAWINGS

The present disclosure will now be described by way of non-limiting examples with reference to the drawings, in which:

FIG. 1A shows an example of a spectrum of an input signal;

FIG. 1B shows the example of a spectrum of the input signal after application of a 6 Hz frequency shift to one channel;

FIG. 2 shows the spectral characteristics of a superposition of two channels of an audio signal containing binaural beats at 6 Hz as perceived by a listener;

FIG. 3 shows an arrangement of a method for generating an output stereo signal containing binaural beats;

FIG. 4 shows an arrangement of a method for generating low-frequency single-offset shifted components of an audio signal;

FIG. 5 shows another arrangement of a method for generating low-frequency multiple-offset shifted components of an audio signal;

FIG. 6 shows another arrangement of a method for generating low-frequency multiple-offset shifted components of an audio signal;

FIG. 7A shows the spectral characteristics of an audio signal containing binaural beats at 6 Hz as perceived by a listener;

FIG. 7B shows the spectral characteristics of an audio signal containing binaural beats at 3.5 Hz as perceived by a listener;

FIG. 7C shows the special characteristics of an audio signal containing binaural beats at 6 Hz and at 3.5 Hz as perceived by a listener;

FIG. 8A shows EEG mappings for a control group (left map) and AWS group (right map) during background baseline measurement;

FIG. 8B shows EEG mappings for a control group (left map) and AWS group (right map) during a reading task;

FIG. 9A shows brain activity map for δ, θ, α, and β brain electrical activity during auditory stimulation after 5 seconds of stimulation;

FIG. 9B shows brain activity map for δ, θ, α, and β brain electrical activity during auditory stimulation after 60 seconds of stimulation;

FIG. 9C shows brain activity map for δ, θ, α, and β brain electrical activity during auditory stimulation after 120 seconds of stimulation;

FIG. 9D shows brain activity map for δ, θ, α, and β brain electrical activity during auditory stimulation after 250 seconds of stimulation;

FIG. 10 shows brain activity exposition for θ and β-waves during a reading activity after auditory stimulation;

FIG. 11 shows changes in power spectrum for brain waves bands and distribution of power spectrum density (PSD) for AWS group before and after stimulation, and after relaxation;

FIG. 12A shows Heart Rate Variability (HRV) Stress Index before and after auditory stimulation for AWS (left plot) and a control group (right plot);

FIG. 12B shows a Stress Index baseline comparison before and after exposure to auditory stimulation in AWS (left plot) and a control group (right plot);

FIG. 13A shows HRV Root Mean Square of Successive Differences (RMSSD) before and after auditory stimulation for AWS (left plot) and a control group (right plot);

FIG. 13B shows HRV RMSSD resultant baseline comparison before and after exposure to auditory stimulation in AWS (left plot) and control group (right plot);

FIG. 14A shows distribution of silence intervals (SI) and phonetic intervals (PI) for fluent speech obtained under reading tasks for control and AWS groups;

FIG. 14B shows distribution of SI and PI for speech with stuttering episodes obtained under reading tasks for control and AWS groups;

FIG. 15A shows speech analysis evaluation before auditory stimulation;

FIG. 15B shows speech analysis evaluation after auditory stimulation;

FIG. 16 shows dynamics of speech quality score for AWS group of participants before stimulation, after stimulation, and post-effect observation after 10 minutes.

### DETAILED DESCRIPTION

Arrangements of the present disclosure generate stimulations in the auditory path. It is known that auditory sensory stimulations may entrain domains of neural steady-state oscillations in certain parts of the cortex (see Schwarz DWF, Taylor P. Human auditory steady state responses to binaural and monaural beats. Neurophysiol Clin (2005) 116(3):658-68. doi:10.1016/j.clinph. 2004.09.014).

Electroencephalography (EEG) and magnetoencephalography (MEG) studies have shown synchronization of brain activity at the frequency of the stimulations and its harmonics in and beyond the corresponding sensory brain areas with visual flicker (see Herrmann, C. S. Human EEG responses to 1-100 Hz flicker: Resonance phenomena in visual cortex and their potential correlation to cognitive phenomena. Exp. Brain Res. 137, 346-353, https://doi.org/10.1007/s002210100682 (2001)), somatosensory tactile stimulation (see Ross, B., Jamali, S., Miyazaki, T. & Fujioka, T. Synchronization of beta and gamma oscillations in the somatosensory evoked neuromagnetic steady-state response. Exp. Neurol. 245, 40-51, https://doi.org/10.1016/j.expneurol.2012.08.019 (2013)) and auditory rhythm (see Ross, B., Miyazaki, T., Thompson, J., Jamali, S. & Fujioka, T. Human cortical responses to slow and fast binaural beats reveal multiple mechanisms of binaural hearing. J. Neurophysiol. 112, 1871-1884, https://doi.org/10.152/jn.00224.2014 (2014)).

Similarly, binaural beats (BB) induce a complex interaction between brain processes (see Oster, G. Auditory beats in the brain. Sci. Am. 229, 94-102 (1973), Pratt, H. et al. A comparison of auditory evoked potentials to acoustic beats and to binaural beats. Hear. Res. 262, 34-44, https://doi.org/10.1016/j.heares.2010.01.013 (2010), Lane, J. D., Kasian, S. J., Owens, J. E., and Marsh, G. R. (1998), Binaural auditory beats affect vigilance performance and mood. Physiol. Behav. 63, 249-252. doi:10.1016/S0031-9384(97)00436, Lavallee, C. F., Koren, S. A., and Persinger, M. A. (2011). A quantitative electroencephalographic study of meditation and binaural beat entrainment. J. Altern. Complement. Med. 17, 351-355. doi: 10.1089/acm.2009.0691, Reedijk, S. A., Bolders, A., and Hommel, B. (2013). The impact of binaural beats on creativity. Front. Hum. Neurosci. 7:786. doi: 10.3389/fnhum.2013.00786, and R. M. Baevsky. Methodical recommendations use kardivar system for determination of the stress level and estimation of the body adaptability standards of measurements and physiological interpretation. 2009.)

Binaural beats occur when both ears of an individual are presented with tones of slightly different frequencies. The frequency difference causes a periodic change in the interaural phase difference. Neurons in the brainstem are sensitive to such interaural phase differences and a binaural beat is perceived as an auditory illusion. Binaural integration at the cortical level leads to the perception of a sound with a single pitch corresponding to the mean of both tones with modulation of the amplitude at a rate equal to the difference between the two tonal frequencies (see Oster, G. Auditory beats in the brain. Sci. Am. 229, 94-102 (1973)).

Brain, or neural, oscillations entrained by the binaural beats have been recorded with EEG and MEG to be similar to the responses elicited by amplitude-modulated sounds at p and θ frequencies (see Pratt, H. et al. A comparison of auditory evoked potentials to acoustic beats and to binaural beats. Hear. Res. 262, 34-44, https://doi.org/10.1016/j.heares.2010.01.013 (2010), and Lavallee, C. F., Koren, S. A., and Persinger, M. A. (2011) A quantitative electroencephalographic study of meditation and binaural beat entrainment. J. Altern. Complement. Med. 17, 351-355. doi: 10.1089/acm.2009.0691).

A challenge with presenting binaural beats in a pure format, i.e. dichotically presenting a listener with a combination of two different pure frequencies, is that the binaural beats may be non-pleasant and irritating to the listener (see Kennel S, Taylor AG, Lyon D, Bourguignon C. Pilot feasibility study of binaural auditory beats for reducing symptoms of inattention in children and adolescents with attention-deficit hyperactivity disorder. J Pediatr Nurs (2010)25(1):3-11. doi:10.1016/j.pedn.2008.06.010). This may be important for modulating hearable frequencies in the j-range of brain activity. In previous studies and popular practical implementations, a single beat of a certain fixed single binaural frequency (e.g., 3, 6, 15-Hz, 40-Hz, etc.) has been used.

Traditionally, binaural beats have been produced by splitting a single frequency audio signal (e.g. a 100 Hz signal) into two channels. A frequency shift is performed on one channel (for example by 18 Hz) which is output to one ear of a listener (i.e. a 118 Hz signal). The original unmodified signal frequency is output to another ear of the listener (e.g. the 100 Hz signal). This results in an 18 Hz binaural beat being presented to the listener. However, it has been identified that it is difficult for a listener (i.e. an individual) to listen to such a tone for an extended period of time.

Arrangements of the present disclosure aim to generate an audio signal containing binaural beats, which is more pleasant for the listener. Such an euphonic audio signal may be used to entrain brain oscillations and are more suited for long-term listening.

Arrangements of the present disclosure aim to induce beta-wave (β-wave) neural oscillations in an individual's brain. This may improve the stability of the speech motor control system in adults with stuttering (AWS), and thus can be used to treat stuttering.

In an arrangement, a method of generating an audio signal containing binaural beats is provided. Instead of introducing binaural beats into a signal of having a single frequency, i.e. a pure tone, binaural beats may be introduced into a signal having a plurality of components of different frequencies. For example, the signal may be a musical work, as described below.

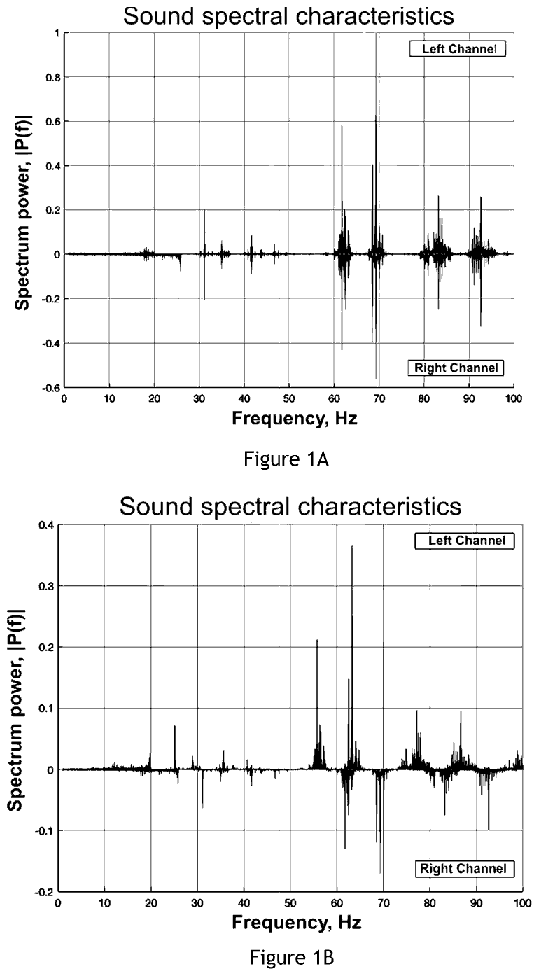

FIG. 1A shows an example of an input audio signal having a plurality of components of different frequency. The example of FIG. 1A shows frequencies up to 100 Hz, however the input audio signal may have a broad range of frequencies that are perceivable by a human. For example, an input audio signal may have a plurality of frequency components in the range between 0 Hz and 20,000 Hz.

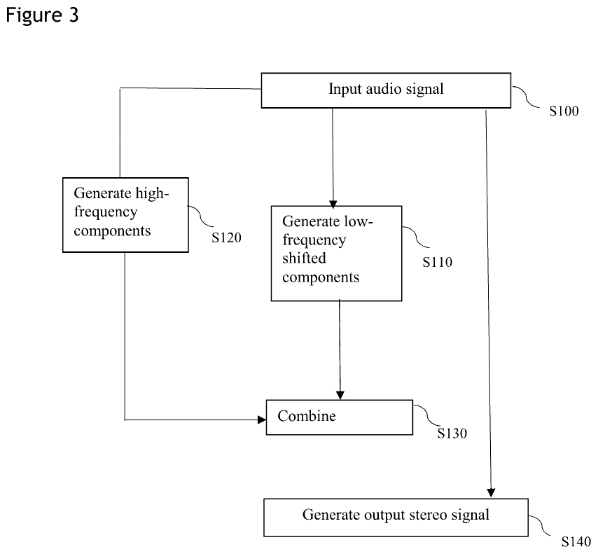

As shown in FIG. 3, in an arrangement the method of generating an audio signal containing binaural beats includes a step of providing an input audio signal S100. As discussed above, the input audio signal includes a plurality of different frequencies present. In an arrangement, the input audio signal a musical work or a musical composition. Preferably, the input audio signal is a pre-recorded sound carrier, i.e. the input audio signal is not an real-time input. Preferably, the input audio signal is a musical work or musical composition which includes vocals. In an arrangement, the tempo of the musical work is between 60 to 80 beats per minute (BPM). Alternatively, the input audio signal may be obtained from nature, such as sounds of a forest, or a flowing river, or sounds of a city such as street noise, or animal sounds, such as the sounds of a cat purring.

In an arrangement, the audio signal has a duration of 5 minutes or more, with at least 30% of the amplitude (i.e. energy density) in a range below a low-pass filter cut-off frequency throughout 80% of the track duration. For example, in an arrangement where a low-pass filter cut-off frequency is 170 Hz, 30% of the amplitude is below this frequency throughout 80% of the track duration.

In an arrangement, the input audio signal is stored in a device memory or a storage device of a computer system configured to generate the audio signal having binaural beats. The input audio signal is obtained by an audio signal input unit.

In an arrangement, the input audio signal is a monophonic signal. In such an arrangement, only a single audio channel is present, meaning that a listener listening to the input audio signal would hear the same audio signal in both ears. In such an arrangement, the step of obtaining the input audio signal S100 involves creating two audio channels xᵢₙ^{left} and xᵢₙ^{right} from the monophonic signal by duplicating the input audio signal, in order to create a stereo input audio signal. The two audio channels created in such a way will be identical.

A first audio channel xᵢₙ^{left} may be played to a left ear of an individual. A second audio channel xᵢₙ^{right} may be played to a right ear of the individual. However, it will be understood that the labels “left” and “right” are merely labels and the first audio channel xᵢₙ^{left} may be played to the right ear, and the second audio channel xᵢₙ^{right} may be played to the left ear.

In an alternative arrangement, the input audio signal may be a stereo audio signal. In such an arrangement, a first channel of the stereo audio input signal may be used as the first audio channel xᵢₙ^{left} and the second channel of the stereo audio signal may be used as the second audio channel xᵢₙ^{right}.

The above-mentioned input audio signal may be represented as an input audio stereo signal xᵢₙ(n), having N samples. The stereo input audio signal may be considered to be a finite input audio stereo signal represented by 2×N discrete samples, where the factor of 2 represents the two audio channels. Each sample may be considered to be a value representing the amplitude of the signal at a particular moment in time. xᵢₙ(n) may be represented as a vector-valued signal sum of left channel and right channel signals:

$${x}_{in} ( n ) = [ {x}_{i n}^{left} ( n ) + {x}_{i n}^{right} ( n ) ] \qquad ( 1 )$$

where n=1 . . . N.

In an arrangement, based on the input audio signal, a first audio signal xₒᵤₜ^{left} and a second audio signal xₒᵤₜ^{left} are generated. For example, the first audio signal may be generated based on the first audio channel xᵢₙ^{left}, and the second audio signal may be generated based on the second audio channel xᵢₙ^{right}. The first audio signal and the second audio signals correspond to the two channels of an output stereo audio signal containing binaural beats.

The first audio signal and the second audio signal have frequency components that are offset relative to one another, which result in the output audio signal containing binaural beats. In other words, frequency components of the first audio signal have a frequency offset relative to corresponding frequency components of the second audio signal. In an arrangement, this is achieved by applying a frequency offset to frequency components of the first audio channel xᵢₙ^{left}, but not applying a frequency offset to corresponding components of the second audio channel xᵢₙ^{right}. Such an example may be seen in FIG. 1B, in which the left channel has been down-shifted by 6 Hz relative to the right channel.

Applying the frequency offset is performed in order to add binaural beats, which is an additional perceived tone, into the output stereo audio signal. Binaural beats can be used to entrain brain waves at a target frequency corresponding to the frequency of the binaural beats. In an arrangement, the binaural beat frequency is selected in the range between 2 Hz and 20 Hz. For example, the binaural beat frequency may be 3 Hz, 6 Hz, 7 Hz, 12 Hz, or 18 Hz.

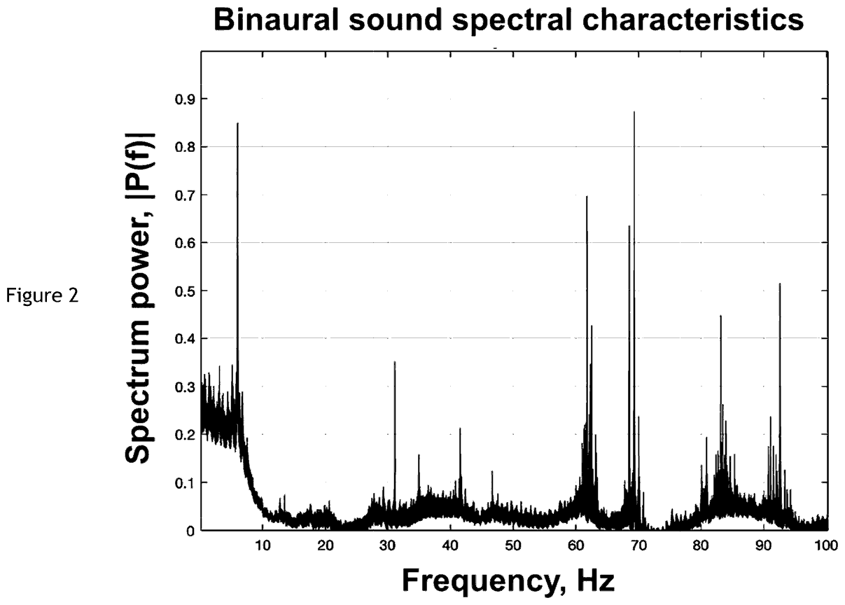

The spectral characteristics of the stereo audio signal containing binaural beats shown in FIG. 1B are shown in FIG. 2. As can be seen in FIG. 2, a large peak is present at 6 Hz, which corresponds to the frequency of the binaural beats, which is present due to the 6 Hz frequency offset which is applied to the left audio channel relative to the right audio channel.

In an alternative arrangement, a frequency offset may be applied to both the first and the second audio channels, as long as a relative frequency offset between the two audio channels is achieved.

In the arrangements as shown in FIGS. 3 to 7, no frequency offset is applied to the second audio channel xᵢₙ^{right}. In such arrangements, a step of generating a second audio signal to be used in the output stereo audio signal is performed by obtaining the second audio channel xᵢₙ^{right} from the input audio signal. In other words, the second audio signal xₒᵤₜ^{right} is the same as the second audio channel of the input audio signal xᵢₙ^{right}.

Preferably, the frequency range in which the frequency offset is present between the first and second audio signals is between 0-250 Hz. For example, the frequency range in which the frequency offset is present between the first and second audio signals may be at frequencies<250 Hz, <230 Hz, <200 Hz, <170 Hz, <150 Hz or <130 Hz. In other words, the frequency range in which the frequency offset is present between the first and second audio signals may be below a cut-off frequency. The typical cut-off frequency may be set between 150-250 Hz, depending on the properties of the audio signal. This has been determined empirically, and may avoid introduction of dissonant or unpleasant high-frequency artefacts that may arise in the resulting output signal having binaural beats.

This may be achieved by applying a frequency offset up to a frequency threshold. A frequency offset may be applied only up to a frequency threshold, i.e. a frequency offset may be applied to only the low frequency range of an audio signal. Optionally, the frequency threshold up to which the frequency offset is applied may be 250 Hz, 230 Hz, 200 Hz, 170 Hz, 150 Hz, or 130 Hz. In an arrangement, the first audio signal xₒᵤₜ^{left} is composed of a high frequency range (or high frequency components) and a low frequency range (or low frequency components). The frequency offset applied to generate the binaural effect may be applied in the low frequency range of the first audio signal. The frequency offset applied to generate the binaural effect may be applied in only the low frequency range of the first audio signal. The terms “high frequency” and “low frequency” are labels to describe the relative locations of the frequency range in which no frequency offset is applied in the first audio signal, and the frequency offset is applied within the first audio signal, respectively. The high frequency range may be defined as the frequency range above the frequency threshold and the low frequency range may be defined as the frequency range below the frequency threshold. In an arrangement, the high frequency components and low frequency components may be determined by the frequency cut-offs of a low-pass filter, and a high-pass filter, which will be described below.

In an arrangement, different processes are performed on the first audio channel xᵢₙ^{left} to generate the high frequency components and the low frequency components of the first audio signal. The first audio channel xᵢₙ^{left} may be first duplicated into two identical copies. A first copy may be subject to the processes to generate the high frequency components, while the second copy may be subject to the processes to generate the low frequency components.

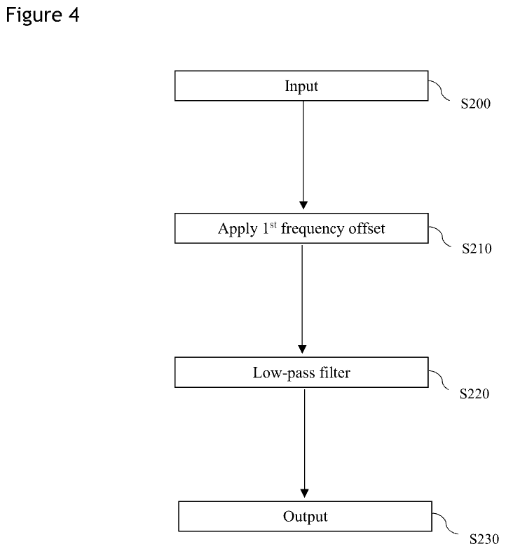

In an arrangement, at step S110 of FIG. 3, low-frequency shifted components x_{lpshift}^{left} of the first audio signal xₒᵤₜ^{left} are generated. As will be discussed in greater detail in relation to FIGS. 4 to 6, generating the low frequency shifted components is performed on one of the copies of the first audio channel xᵢₙ^{left}. Generating the low-frequency shifted components based on the first audio channel involves applying a frequency offset shift to the first audio channel, and low-pass filtering the first audio channel. The output of step S110 is a low-frequency region of the first audio channel having a frequency offset relative to a corresponding low-frequency region of the second audio signal.

In an arrangement, at step S120 of FIG. 3, high-frequency components xₕₚ^{left} of the first audio signal xₒᵤₜ^{left} are generated. This may be performed using a high-pass filter. In an arrangement, the frequency of the high-pass filter is set to the same frequency as a cut-off frequency of a low-pass filter used in step S110 described later. Step S120 may preserve the musical component of the audio input signal in the high-frequency region, which results in a more pleasant output audio signal. This also avoids a frequency shift being present in the high frequency range which would otherwise result in unpleasant or high-frequency dissonant sounds that would be unpleasant to the human ear.

In an arrangement, a high-pass filter of order N=200, with a cut-off frequency of 170 Hz may be applied follows:

$${x}_{hp}^{left} ( n ) = {\sum }_{k = 0}^{N} {b}_{k} {x}_{i n}^{left} ( n - k ) \qquad ( 2 )$$

In equation (2), bₖ are the coefficients of a FIR high-pass filter, and xₕₚ^{left}(n) represents the high-pass (hp) filtered input audio signal.

At step S130 of FIG. 3, the first audio signal xₒᵤₜ^{left} is generated and is output. The first audio signal is generated by combining the low-frequency shifted components x_{lpshift}^{left} of the first audio signal and the high-frequency components xₕₚ^{left} of the first audio signal. In an arrangement, combining these signals involves summing together the two signals. For example, the first audio signal may be obtained as follows:

$${x}_{out}^{left} ( n ) = {x}_{hp}^{left} ( n ) + {x}_{lpshift}^{left} ( n ) \qquad ( 3 )$$

The resulting first audio signal is output to a stereo output signal generating unit so that the first audio signal xₒᵤₜ^{left} may be incorporated into the output audio signal.

In an arrangement, the step of applying a frequency offset to components of the first audio channel is performed in the frequency domain. In an arrangement, the remaining steps, such as generating high-frequency components in step S110, applying low-pass filtering to the shifted frequency components, and combining the low-frequency shifted components and the high-frequency components may be performed in the temporal domain.

Alternatively, all of the steps may be performed in the frequency domain, including applying the high and low pass filters, and the step of combining the low-frequency shifted components and the high-frequency components. For example, at step S100 when the input audio signal is obtained, a frequency representation of the input audio signal may be obtained by applying a Fourier transform to the input audio signal. The resulting frequency domain representation of the input audio signal may then be used in steps S110, S120, and S130. In other arrangements, some of the steps, including applying the frequency offset, are performed in the frequency domain, and the remaining steps are performed in the temporal domain.

At step S140, the first audio signal xₒᵤₜ^{left} and the second audio signal xₒᵤₜ^{right} are used to generate the output audio signal containing binaural beats. The output audio signal is a stereo audio signal in which the first audio signal is played on a first channel of the output signal, and the second audio signal is played on a second channel of the output signal, such that a listener listens to the first audio signal in one ear, and the second audio signal in another ear. As a result, the binaural beats will be heard by the listener. This may be achieved by the listener wearing earphones or headphones.

In an arrangement, at step S140, the stereo output audio signal containing binaural beats is normalised to a predetermined audio volume. The volume may be a predetermined decibel (dB) level. For example, the output audio stimulus may be normalised at 55 dB to provide a comfortable effective level of auditory stimulation. In an arrangement, normalization can be performed according to the maximum and minimum amplitudes of all the binaural components.

The normalized amplitude of the output signal can be described by the following formula:

$${A}_{L} = \frac{X - {X}^{min}}{{X}^{max} - {X}^{min}} \qquad ( 4 )$$

where X^{min} is a minimal value, and X^{max} is a maximal value defined by:

$$( 5 )$$

$${X}^{min} = min \{ {X}^{1} , {X}^{2} , {X}^{3} , … , {X}^{n} \} , \qquad {X}^{max} = max \{ {X}^{1} , {X}^{2} , {X}^{3} , … , {X}^{n} \}$$

where n is a number of binaural components of the mixture.

The second audio signal may be normalized according to the equation:

$${A}_{R} = \frac{{X}_{R} - {X}^{min}}{{X}^{max} - {X}^{min}} \qquad ( 6 )$$

Other normalisation techniques, such as peak normalisation, loudness normalisation in accordance with EBUR128 standard, or others, may be applied to the output audio signal instead of the above-mentioned normalisation technique.

The output audio signal may be generated by a stereo output audio signal generating unit. The stereo output audio signal may be stored in a device storage or memory for retrieval and playback at a later time.

In an arrangement, various parameters of the stereo output audio signal containing binaural beats may be modified (i.e. personalised) in order to optimise the desired effect on an individual based on physiological data. In an arrangement, the tempo of the output audio signal may be changed. In such an arrangement, the tempo may be changed in order to match a physiological measurement of the individual that will be subject to the output audio signal stimulus, such as their heart rate. For example, the specific target frequencies of the binaural beats, the number of different binaural beat frequencies, and their relative amplitude may be adjusted based on individuals physiologic data. For example, a measurement with any suitable apparatus may be performed of the heart rate of the individual, and then the output signal tempo may be changed to synchronise with the individual's measured heart rate as measured by a consumer wearable ECG device. Such a technique makes use of the known effect of heart rate synchronisation with music tempo. Due to the impact of the musical influence with a tempo in the physiological range between 60 and 80 BPM, for example, it is possible to achieve temporal synchronisation of rhythms, e.g. for time periods of up to 30 seconds. This effect may push the Autonomic Nervous System (ANS) to a parasympathetic state, in order to reduce stress levels and increase the level of heart rate variability (HRV). Reducing stress and increasing HRV may contribute to reducing stress and may assist in sleep stimulation, reducing stuttering, and promoting relaxation.

The methods of generating the low-frequency shifted components of the first audio channel will now be described with reference to FIGS. 4 to 6.

In an arrangement as shown in FIG. 4, a single frequency offset is applied to the first audio channel. At step S200, the copy of the first audio channel from the input audio signal is provided.

At step S210, a frequency offset is applied to the first audio channel. In an arrangement, the frequency offset is applied to the whole frequency range of the first audio channel. For example, the frequency offset will be applied both to the high-frequency range and the low-frequency range of the first audio channel. However, in an alternative arrangement, the frequency offset may be performed on only a low-frequency range of the first audio channel. For example, a frequency offset may be applied to the first audio channel only up to a frequency threshold. Thus, components of the first audio signal up to a frequency threshold may have a frequency offset relative to corresponding components of the second audio signal. Such a low-frequency range may be obtained by low-pass filtering the first audio channel before applying the frequency offset.

In an arrangement, applying the frequency offset is performed in the frequency domain. The first audio channel has a spectrum in the frequency domain of

$${X}_{i n}^{left} ( k ) = ℱ [ {x}_{i n}^{left} ( n ) ] \qquad ( 7 )$$

where [symbol] is the Discrete Fourier Transform (DFT) operation, which is commonly evaluated at the frequencies

$$f = \frac{k}{N}$$

as following:

$${X}_{i n}^{left} ( k ) = {\sum }_{n = 0}^{N - 1} {X}_{i n}^{left} ( n ) {e}^{- i \frac{2 π kn}{N}} , k = 0 , … , N - 1 \qquad ( 8 )$$

The frequency offset, f_{offset}, may then be applied to the first audio channel in the frequency domain in order to generate a shifted first audio channel. For example, the frequency offset, f_{offset}, is applied as either

$${X}_{shift}^{left} ( k ) = {X}_{i n}^{left} ( k ) - {f}_{offset} N \qquad ( 9 )$$

or

$${X}_{shift}^{left} ( k ) = {X}_{i n}^{left} ( k ) + {f}_{offset} N \qquad ( 10 )$$

In an arrangement, the frequency offset, f_{offset}, is between 0 and 22 Hz. The specific frequency of the frequency offset may be determined in advance. In an arrangement, the frequency offset may be determined in dependence on data relating to speech quality metrics of an individual, which will be described later.

In an arrangement, after applying the frequency offset, the shifted signal X_{shift}^{left}(k) is transformed back to the temporal or time domain. The shifted signal in the frequency domain left channel may be transformed back to the time domain using Inverse DFT:

$${x}_{shift}^{left} ( n ) = {\sum }_{k = 0}^{N - 1} {X}_{out}^{left} ( k ) {e}^{- i \frac{2 π k}{N} n} , n = 0 , … , N - 1 \qquad ( 11 )$$

At step S220, after applying the frequency offset to the first audio channel, a low pass filter is applied to x_{shift}^{left}. As described above, the low-pass filter is applied to cut off frequencies above a certain frequency which may prevent unpleasant (dissonant) high-frequency artefacts that may arise due to the binaural processing. Additionally, the low-pass filter may remove unpleasant audio effects such as reverberation caused by the application of the binaural frequency shift at high frequencies.

In an arrangement, the low-pass filter may be applied at a frequency of up to 250 Hz. For example, the cut-off frequency of the low-pass filter may be 170 Hz. A frequency of 170 Hz may achieve result in good euphonic binaural beats being introduced into the input audio signal. A lower cut-off frequency may be more effective in preventing dissonant artefacts but may lead to deterioration of sound quality. In other arrangements, the cut-off frequency of the low-pass filter is selected based on the spectral distribution of the input audio signal in the range of 0-250 Hz.

In an arrangement, the cut-off frequency of the low-pass filter may be determined based on the last significant frequency in the frequency range up to 250 Hz. For example, in an arrangement, in the frequency range from 150 to 250 Hz, the spectrum of the first audio channel is obtained, and the frequency having the highest amplitude is identified. This frequency may be used as the cut-off frequency of the low-pass filter.

In an arrangement, the low-pass filter may be a Finite Impulse Response (FIR) filter of order N=200 with passband frequencies 0-170 Hz applied

$${x}_{lpshift}^{left} ( n ) = {\sum }_{k = 0}^{N} {b}_{k} {x}_{shift}^{left} ( n - k ) \qquad ( 12 )$$

where bₖ are the coefficients of the FIR band-pass filter. The resulting signal x_{lpshift}^{left} is limited to the desired frequency range of the first audio signal xₒᵤₜ^{left}.

At step S230, the resulting signal x_{lpshift}^{left} is output as the low-frequency shifted components of the first audio signal. The resulting signal x_{lpshift}^{left} corresponds to the output of step S110 in FIG. 3, and is provided to be combined with the high-frequency components generated in step S120 to form the first audio signal xₒᵤₜ^{left} in step S130.

As previously described, in some arrangements, only the step of applying the frequency offset is performed in the frequency domain. In these arrangements the first audio channel xᵢₙ^{left} is Fourier transformed before applying the frequency offset in the frequency domain, and then inverse-Fourier transformed after application of the frequency offset. However, in other arrangements, as described above, the steps S200, S210, S220, and S230 may be all be performed in the frequency domain. In these arrangements, the Fourier transform may be performed as part of step S100, and the input at step S200 may already be in the frequency domain.

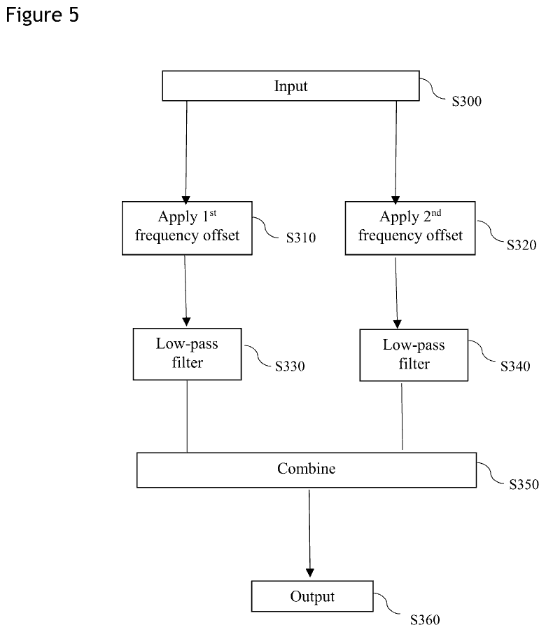

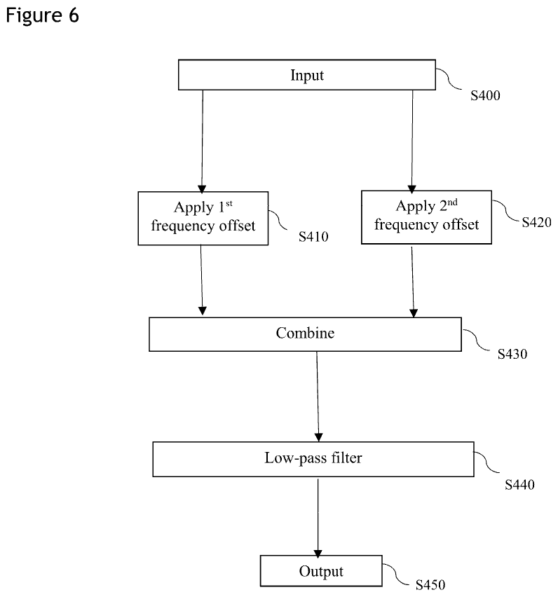

In an arrangement, for example as shown in the examples of FIGS. 5 and 6, a plurality of different frequency offsets are applied to the first audio channel xᵢₙ^{left}. As shown in FIGS. 5 and 6, two frequency offsets are applied, however in other arrangements more than two frequency offsets are applied. FIGS. 5 and 6 show arrangements of the processing that occurs in step S110 of FIG. 3.

As shown in FIGS. 5, the step of generating low-frequency shifted components of the first audio signal xₒᵤₜ^{left}, includes a step S300 of obtaining a copy of the input audio channel xᵢₙ^{left}. In an arrangement, applying the plurality of frequency offsets is performed on a separate copy of the input audio channel obtained at step S300. Step 300 therefore includes obtaining a plurality of copies of the input audio channel xᵢₙ^{left} are generated, each copy to be used for the application of a different frequency offset. The copies may be obtained by duplicating the input audio channel to create the plurality of identical copies.

At step S310, a first frequency offset is applied to the input audio channel xᵢₙ^{left}. At step S320, a second frequency offset is applied to the input audio channel xᵢₙ^{left}. The first and second frequency offset are different. In arrangements with additional frequency offsets to be applied, a third frequency offset may be applied, and so on etc. The steps S310 and S320 correspond to step S210 as described in relation to FIG. 4, and the details are not repeated. The frequency offset applied in steps S310 and S320 is between 0 and 22 Hz, with a first frequency in this range being applied as the first frequency offset applied in step S310, and a different, second frequency in this range being applied as the second frequency offset applied in step S320.

At step S330 a low-pass filter is applied to the first audio channel after application of the first frequency offset. At step S340 a low-pass filter is applied to the first audio channel after application of the second frequency offset. The low-pass filters applied at steps S330 and S340 may be identical, and have the same cut-off frequency. Steps S330 and S340 correspond to the low-pass filter step S220 described in relation to FIG. 4 and the specific details are not repeated.

The outputs of steps S330 and S340 are low-frequency shifted components of the first audio signal at the first frequency offset and at the second frequency offset, respectively. At step S350, these outputs are combined together to form the low-frequency shifted components of the first audio signal. In an arrangement, these outputs are combined by averaging them together (e.g. summing together the signal intensities at each frequency for each frequency offset signal and dividing by the number of signals). The resulting signal will include frequency offset components corresponding to the first frequency offset and the second frequency offset.

At step S360, the resulting signal is output from the step of generating the low-frequency shifted components of the input audio signal xₒᵤₜ^{left}, in a similar manner to step S230.

The example of FIG. 6 is similar to the example of FIG. 5 as described above. Steps S400, S410, S420, and S450 correspond directly to steps S300, S310, S320, and S360 as described above, respectively. In the arrangement as shown in FIG. 6, the step S430 of combining, or averaging the signals after application of first and second frequency offsets is performed before applying the low-pass filter at step S440. The step S430 otherwise corresponds directly to step S350.

In other words, step S430 in which the frequency offset signals are combined is performed across the whole frequency spectrum, and not the low-frequency spectrum which is obtained by the low-pass filters at steps S330 and S340.

After step S430, the combined signals including frequency offset components corresponding to the first frequency offset and the second frequency offset is low-pass filtered at step S440. The low-pass filtering step is the same as that described for step S220 above.

As discussed in relation to the arrangements as shown in FIG. 4, the first frequency offset and the second frequency offset may be determined on the basis of data relating to speech quality metrics of an individual. The first frequency offset and the second frequency offset may be determined by a first frequency offset determination unit and a second frequency offset determination unit, respectively.

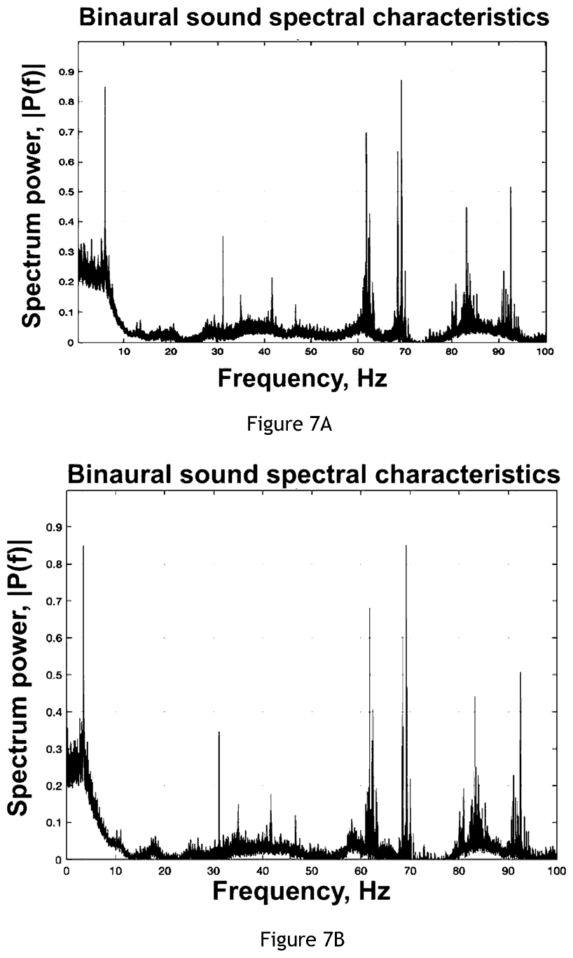

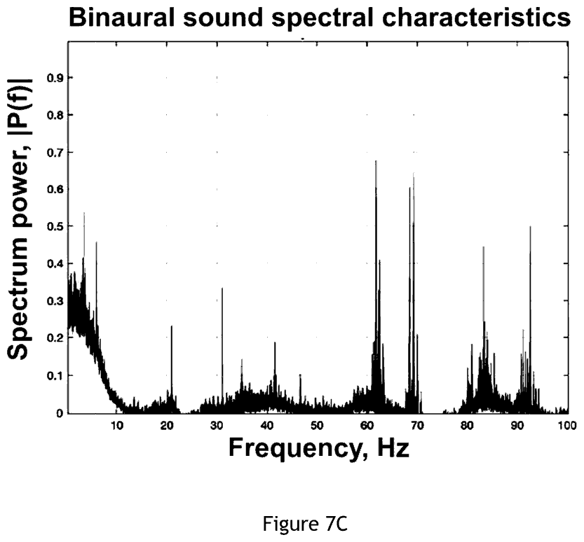

FIGS. 7A, 7B, and 7C show the spectral characteristics of an audio signal containing binaural beats at different frequencies, as perceived by a listener. For example, FIG. 7A shows the spectral characteristics, as perceived by a listener, for an audio signal containing binaural beats, for which a single frequency offset at 6 Hz has been applied. FIG. 7B shows the spectral characteristics, as perceived by a listener, for an audio signal containing binaural beats, for which a single frequency offset at 3.5 Hz has been applied. On the other hand, FIG. 7C shows the spectral characteristics, as perceived by a listener, for an audio signal containing binaural beats, for which two different frequency offsets, at 6 Hz and at 3.5 Hz have been applied (for example, using the methods shown in FIG. 5 or 6). As can be seen in FIG. 7C, not only is there a perceived tone (i.e. a binaural beat) at 6 Hz and at 3.5 Hz, but there is also another perceived tone (a binaural beat) at 21 Hz, corresponding to a superposition of the 6 Hz and 3.5 Hz binaural beats.

In many applications, including sleep or stuttering therapy, it may be advantageous to create binaural beats at multiple frequencies in order to trigger or stimulate the listener's brain at several different frequencies. For example, one or more of alpha (α), beta (β), delta (δ), and/or theta (θ) brain frequencies may be modulated simultaneously. This may be achieved by introducing a plurality of frequency offsets into the first audio signal as described above.

Creating binaural beats at multiple different frequency offsets (e.g. at 6 Hz and at 3.5 Hz) has a similar response in the brain as a single frequency offset corresponding to the superposition of those different frequency offsets (e.g. at 21 Hz). This is because the multiple different frequencies become superposed with one another in the brain, so that the binaural frequency shifts are multiplied. As a result, in a situation where a binaural beat at 21 Hz is desired for optimal brain wave entrainment, the same result can be achieved with the lower frequency 3.5 Hz and 6 Hz frequencies. This results in an output stereo audio signal containing binaural beats which are more pleasant to listen to, due to the deeper frequencies, and with less interference with the musical content of the audio signal.

The above-described methods may be implemented on any suitable computer system configured to perform each of the described steps, or on computer-readable software that can be executed by a computer system. The computer program product may include all the features enabling the implementation of the methods and functions described herein, and which when loaded in a computer system is able to carry out these methods and functions. Terms such as computer program, software program, program, program product, software, etc., in the present context mean any expression, in any language, code or notation, of a set of instructions intended to cause a system for synthesizing binaural beats stimuli for mental state modulation and speech fluency improvement.

The computer system for performing the described methods may be implemented by one or more computing devices. The computing devices may include one or more client or server computers in communication with one another over a near-field, local, wireless, wired, or wide-area computer network, such as the Internet, and at least one of the computers is configured to receive signals from sensors worn by a user. In an implementation, the sensors include one more bio-signal sensors, such as electroencephalogram (EEG) sensors, galvanometer sensors, electrocardiograph (ECG) sensors, heart rate sensors, eye-tracking sensors, blood pressure sensors, pedometers, gyroscopes, and any other type of sensor. The sensors may be connected to a wearable computing device, such as a wearable headset or headband computer worn by the user. The sensors may be connected to the headset by wires or wirelessly. The headset may further be in communication with another computing device, such as a laptop, tablet, or mobile phone such that data sensed by the headset through the sensors may be communicated to the other computing device for processing at the computing device, or at one or more computer servers, or as input to the other computing device or to another computing device. The one or more computer servers may include local, remote, cloud based or software as a service platform (SAAS) servers. Embodiments of the system may provide for the collection, analysis, and association of particular bio-signal and non-bio-signal data with specific mental states for both individual users and user groups. The collected data, analyzed data or functionality of the systems and methods may be shared with others, such as third party applications and other users via API. Connections between any of the computing devices, internal sensors (contained within the wearable computing device), external sensors (contained outside the wearable computing device), user effectors, and any servers may be encrypted. Collected and analyzed data may be used to build a user profile that is specific to a user.

In an arrangement, an individual's speech quality may be measured using metrics of phonetic intervals (PI) and silence intervals (SI). A PI corresponds to the duration of one phoneme, and SI corresponds to the time interval between neighbouring phonemes. Detection of an individual's PI and SI may be used to evaluate their speech quality, and may be used to monitor changes in speech quality over time. In an arrangement, an individual's speech quality may be used to evaluate the presence of stuttering in the individual. Typically, it is observed that adults with stuttering (AWS) have longer SI, and usually the PI median value is shifted to the values in the range of between 150-259 ms (i.e. more longer durations of phonemes). Typically, the total number of PI as well as SI is lower in the AWS group (i.e. less number of total words spoken).

Detection of an individual's PI and SI may be performed based on an adaptive thresholds approach. In an arrangement, this may be performed based on a sample of the individual's speech. The sample may be a recording taken and stored in advance, or may be obtained live from the individual.

In an arrangement, the sample may be based on an individual reading a suitable text such as a text with a predetermined number of words (usually between 150-250 words) comprising a variety of different words.

In an arrangement, a first stage involves filtering the sample of the individual's speech to a bandwidth of between 60 and 1200 Hz. This may reduce or eliminate other frequencies, noises, and/or artefacts outside of this range which could interfere with a correct assessment of the speech. The filtered sample is then squared to obtain temporal changes of absolute intensity.

Next, the individual's speech is analysed in two stages with a sliding 1 second window. A 1 second window is selected based on the average phoneme duration in speech. A different window may be selected, as long as it is not too long in order not to miss out in very fast changes in voice and phonemes, but also not too short so as to not count additional phonemes in each window. A time period of 10 seconds at the beginning of the sample is used to calculate an average signal intensity of speech and silence, in order to determine the amplitude of the useful signal and noise level. Next, phonetic intervals may be quantified together with silence intervals between them.

In an arrangement, the adaptive threshold value for phonemes is determined by the formula:

$${l}_{thr} = ( {Max}_{i} - {Mean}_{i} ) \times α + {Mean}_{i} \qquad ( 13 )$$

where i=1 . . . N is a number of windows, Maxi is the maximal value of signal within the i^{th} window, Meanᵢ is the mean average value of the signal within the i^{th} window, and a is a peak power factor (α<1.0). Such an arrangement may enable a robust and accurate processing of signals with variable amplitude and temporal characteristics, because the threshold intensity value is automatically adjusted to the input signal.

After the detection of PI, a speech quality score (SS) can be calculated as a normalized median of PIs:

$$SS = \frac{median ( PI )}{T} \qquad ( 14 )$$

where median(PI) is the median value of PIs (in seconds) and Tis the total duration of detected PIs (in seconds). From the determined PI and SI of an individual, it is possible to evaluate the total number of PI and SI, a maximum bin of PI or the phonetic peak, a maximum PI value, and a maximum SI value.

In an arrangement, an individual's speech quality score may be determined before and after listening to an audio signal containing binaural beats. The audio signal containing binaural beats may be an audio signal determined according to previously known techniques, or may be the audio signal determined according to the above-described arrangements.

Each individual's brain waves are different, and therefore a response to listening to the output audio signal discussed above may differ between individuals. Additionally, each individual's brain may also learn over time, which may desensitize the individual to the BB-containing audio track.

In some arrangements, the desired frequency offset for targeting β-wave activation in an individual's brain is in the range form 15 Hz to 22 Hz. However, in other arrangements this is not the case, and may vary from individual to individual. In some arrangements, the specific range is adjusted for each individual based on the optimal binaural beat frequency for each individual.

In general, β-waves in the brain are stimulated by the presence of binaural beats in the region of between 15 Hz to 22 Hz. However, not every individual has the same response to a particular frequency (e.g. 18 Hz), and may have a greater response at a slightly different frequency. Therefore, the exact frequency for stimulation in the beta brain wave range may be changed, and may be determined based on data obtained from the individual.

In an arrangement, an individual's electrical brain activity may be detected using electroencephalography (EEG). Such a technique involves playing an output audio signal containing binaural beats at a first binaural beat frequency to the individual, and measuring the degree of beta wave entrainment in the brain. Then, the frequency of the binaural beats in the audio signal may be altered, and measured again. In an arrangement, an individual may listen to an audio signal containing binaural beats at a different frequencies in the range of 0 to 22 Hz, whilst the individual's brain activity is monitored. In dependence on the individual's brain activity, the optimum frequency to achieve β-wave stimulation in the individual's brain may be identified, and may be used as the frequency offset in the methods of generating an output audio signal containing binaural beats. However, a sufficiently high-quality EEG is a complex and expensive medical device that requires a certain level of skills to work. Therefore it may be difficult for a consumer to optimise the brain waves in this manner.

In another arrangement, the calibration and optimisation of the binaural beat frequencies may be performed using voice recording. As discussed above, an individual's PI and SI are measured before and after auditory stimulation using the binaural beats audio signal. Each round of auditory stimulation may be performed with a slightly different binaural beat frequency, and the PI and SI before and after may be measured. After several rounds of the auditory stimulation, it is possible to assemble a dataset comprising the associated speech quality scores before and after auditory stimulation (from the PI and SI), as well as the specific frequencies of the binaural beats.

In an arrangement, it is possible to use machine learning techniques to optimise the binaural beat frequencies in order to select an optimal frequency for stimulation in order to maximise the improvement in speech quality.

The frequency offset or frequency offsets used when generating the output audio signal containing binaural beats, as well as input audio signal, can be selected to maximize the desired physiological effect based on individual and population-wide data by applying machine learning of the recommendation system type, such as content based filtering (CBF) (see Thorat, Poonam B., R. M. Goudar, and Sunita Barve. “Survey on collaborative filtering, content-based filtering and hybrid recommendation system.” International Journal of Computer Applications 110.4 (2015): 31-36) or collaborative filtering (CF) (see Su, Xiaoyuan, and Taghi M. Khoshgoftaar. “A survey of collaborative filtering techniques.” Advances in artificial intelligence 2009 (2009).)

In such arrangements, listeners are presented with multiple signals randomly, and physiological effects will be recorded. The features of input audio signal (such as music genre or type of natural sound, artist, instruments used, etc.), the frequency offsets for the binaural beats, and physiologic effects and individual's demographic data (age, gender, native tongue, etc) may be used as inputs for algorithm training.

In an arrangement using content based filtering applied to signal customization, each individual may be presented with several randomly selected frequencies in the target range for beta-wave entrainment (e.g. 16 Hz, 18 Hz, 19 Hz), and the individual's physiological response is recorded. Then, a second-order curve may be fitted and a maximum may be inferred. Subsequently, the user may be presented with the stimulus with the inferred optimum frequency, physiological endpoint recorder, and the model is updated to provide a refined optimal frequency. The process may be repeated multiple times to refine the target frequency.

Additionally, the choice of the input audio signal may be optimized using a collaborative filtering procedure such as factorization machines (see [Thorat 2015] referenced above), with user physiological endpoints or rating responses to various carrier tracks serving as inputs. This may optimise for physiological endpoints (e.g. fluency, heart rate, HRV, or EEG derived metrics).

In an arrangement, stuttering may be treated using an audio signal containing binaural beats. Stuttering may be treated using any audio signal containing binaural beats, including audio signals generated in accordance with traditional techniques. In preferred arrangements, the audio signal containing binaural beats is generated according to the arrangements described above, which result in a more euphonic and pleasant audio signal that an individual will enjoy listening to for an extended period of time. This will increase the length of time that the individual may tolerate listening to the audio signal, and assist in treating the stuttering.

In the following section, experimental data is provided supporting the premise that performing an audio signal generated in accordance with the above-described methods, comprising binaural beats, may be used to reduce the effects of stuttering in AWS.

In accordance with arrangements of the present disclosure, adult volunteers with stuttering (5 males aged 18-33 years; mean age: 26.6 years) and fluently speaking volunteers (5 males aged 25-33 years; mean age: 28.2 years) signed an informed consent and participated in the study at the laboratory of Physics, Electronics and Computer Systems department of Dnipro National University (institutional review board protocol #15-05/21, from 2021-05-10). All participants were right-handed. All control participants reported no history of speech, language or hearing difficulties.

Auditory stimuli obtained in accordance with the above-described arrangements were applied for a duration of 5 minutes. The experiment protocol consisted of six stages:
(1) baseline measurement in a relaxed state with participants in a closed-eyes state at rest,
(2) reading task activity before stimulation,
(3) auditory binaural beats stimulation in accordance with the above-described arrangements,
(4) reading task activity after the stimulation,
(5) reading task activity after a further 10 minutes of relaxation, and
(6) final measurement to discover changes in baseline state, again a closed-eyes state at rest.

During each of the measurements, electroencephalography (EEG) and electrocardiograph (ECG) signals were continuously recorded with certified medical equipment and desktop companion software. Voice was recorded with a built-in microphone of the Laptop MacBook Pro 13′ (2015 y).

For each participant, their baseline ECG to estimate Heart Rate Variability (HRV) parameters and EEG was recorded at the beginning of the session for 5 minutes in resting conditions with closed eyes with no external stimuli. Next, every participant read a standardized text in their native language for 5-minutes. The speech was recorded and subsequently analysed.

A medical-grade digital brain electric activity monitor (CONTEC KT88-3200, S/N 591012399249) was used to collect EEG raw data with conventional wet cap-electrodes. 10/20 EEG electrodes were used and placed according to the international standard system. The 10/20 system or International 10/20 system is an internationally recognized method to describe and apply the location of scalp electrodes in the context of an EEG exam, polysomnograph sleep study, or voluntary lab research. This method was developed to maintain standardized testing methods ensuring that a subject's study outcomes (clinical or research) could be compiled, reproduced, and effectively analyzed and compared using the scientific method. The system is based on the relationship between the location of an electrode and the underlying area of the brain, specifically the cerebral cortex. Reference: Jasper, Herbert H. (May 1958). “Report of the committee on methods of clinical examination in electroencephalography”. Electroencephalography and Clinical Neurophysiology. 10 (2): 370-375. doi:10.1016/0013-4694(58)90053-1. Digital signal filtration systems (such as those described in Anshul, D. Bansal and R. Mahajan, “Design and Implementation of Efficient Digital Filter for Preprocessing of EEG Signals,” 2019 6th International Conference on Computing for Sustainable Global Development (INDIACom), 2019, pp. 862-868) were applied to decrease moving artefacts and external electromagnetic interference (EMI) noise. Power spectrum density (PSD) and spatial distribution of brain excitation (2D BEAM, numerical BEAM), compressed spectrum graph, and trend graph were analysed using BrainTest v3.1.5.0 software. Output raw data was saved into the European EDF+ format.

The sampling rate for each channel was 200 Hz with 0.1 μV resolution and a 0.016-1000 Hz input signal frequency range. The input data was digitally filtered with a f=0.1-30 Hz band pass filter to exclude electromyographic (EMG) artifacts. Brain spectral activity was evaluated using Fast Fourier Transform (FFT) and PSD. EMI noise was reduced by applying a notch filter with a cut-off frequency of 50 Hz. Other noises, such as electrode displacement disturbances, were also excluded from the analysis. Absolute power was numerically integrated over each frequency band including δ (0.5-4 Hz), θ (4-7 Hz), α (7-13 Hz), β (13-30 Hz). These calculations were conducted over every electrode's position (24-electrode EEG measurement). Consequently, the average of 24 channels was used for statistical analysis of brain wave power. Spatial distribution of PSD was visualized as heatmaps for each EEG spectral band (BEAM representation). PSD function for EEG signal was calculated using a standard formula (see Alam, R.-u.; Zhao, H.; Goodwin, A.; Kavehei, O.; McEwan, A. Differences in Power Spectral Densities and Phase Quantities Due to Processing of EEG Signals. Sensors 2020, 20, 6285. https:/doi.org/10.3390/s20216285) for discrete time-domain signal.

For electrocardiographic (ECG) recordings, we used a CONTEC 8000GW (HW S/N:39124881923) with four wet electrodes, which were placed on the limbs to obtain a conventional ECG six lead system (I, II, III, avL, avRand avF).

To provide stability of measurements of ECG lead-off detection anti-aliasing, baseline drift decreasing functions were applied. ECG raw data passed through the digital filtration tract.

with a passing band f=0.1-30 Hz band pass filter to exclude EMG artifacts. EMI influence is reduced by the application of a notch filter with cut-off frequency fc=50 Hz. ECG raw data exported to the HL7 aECG standard format (.xml), convenient for data sharing, academic exchange and further analysis with external tools (signal digital filtration, RR intervals detection and HRV analysis).

Autonomic nervous system (ANS) state and the stress level is assessed by heart beat-to-beat (RR intervals) variability of participants using set of standard ECG HRV parameters with respect to the European Society of Cardiology and North American Society HRV Standard (see Malik, Marek. (1996). Heart rate variability: Standards of measurement, physiological interpretation, and clinical use. Circulation. 93. 1043-1065): meanRR, meanHR, SDNN, RMSSD, LF/HF, Stress Index. All parameters were measured during each experiment session, described before.

The stress index (SIdx) or Baevsky's stress index was computed according to the formula (see Reedijk, S. A., Bolders, A., and Hommel, B. (2013). The impact of binaural beats on creativity. Front. Hum. Neurosci. 7:786. doi: 10.3389/fnhum.2013.00786):

$$SIdx = \frac{AMo \times 100 \%}{2 Mo \times MxDMn} \qquad ( 15 )$$

where AMo is the so-called mode amplitude presented in percentage points, Mo is the mode (the most frequent RR interval) and MxDMn is the variation scope reflecting the degree of RR interval variability. The mode Mo is simply taken as the median of the RR intervals. The AMo is obtained as the height of the normalised RR interval histogram (bin width 50 ms) and MxDMn as the difference between longest and shortest RR interval values. In order to make SIDx less sensitive to slow changes in mean heart rate (which would increase the MxDMn and lower AMo), the very low frequency trend is removed from the RR interval time series by using the smoothness priors method.

The auditory stimulation was provided in accordance with the arrangements described above, and the speech quality was also measured in accordance with the embodiments described above.

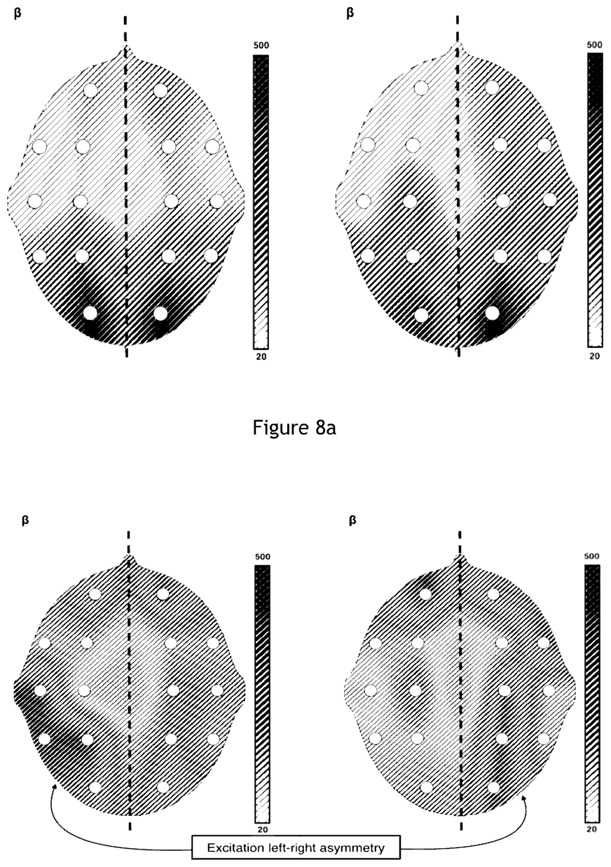

The averaged and normalized results of neural activity during reading activity for two groups shown in FIG. 8A. No significant difference of PSD and its asymmetry in 0-spectrum between the control group and the AWS group was observed in the baseline state. The averaged and normalized results of neural activity during reading activity for two groups are shown on FIG. 8B.

There is a measurable quantitative difference between the distribution of cortex β-activity stutters compared to the non-stutters. An average PSD of 3-waves is 51.1±20.1 μV²/Hz in AWS group and 92.4±23.2 μV²/Hz in the control group. It should be noted that asymmetry in the distribution of β-excitation in the left and right brain hemisphere was found for the AWS group. For this study, a lower spectral density of β-waves for the AWS was observed at the left temporal cortex (T7, TP7, P7), parietal cortex (C3, CP3, P3) and ventrolateral prefrontal cortex (F7) compared to the left-hemisphere activity at the control group. The mean coefficient of asymmetry in the AWS group is −15.7±43.7 and for the control group is 17.1±27.1. This fact is confirmed by the results obtained in Özge, A., Toros, F., and Çömelekoǧlu, Ü. (2004). The role of hemispheral asymmetry and regional activity of quantitative EEG in children with stuttering. Child Psychiatry Hum. Dev. 34, 269-280. doi: 10.1023/B:CHUD.0000020679.15106.a4.

Asymmetrical excitation may be due to the β-activity deficit during speech disfluency at Wernicke's area around electrodes positions C3, CP3, P3 and Broca's area around electrode position F7 being responsible for speech recognition and pronunciation.

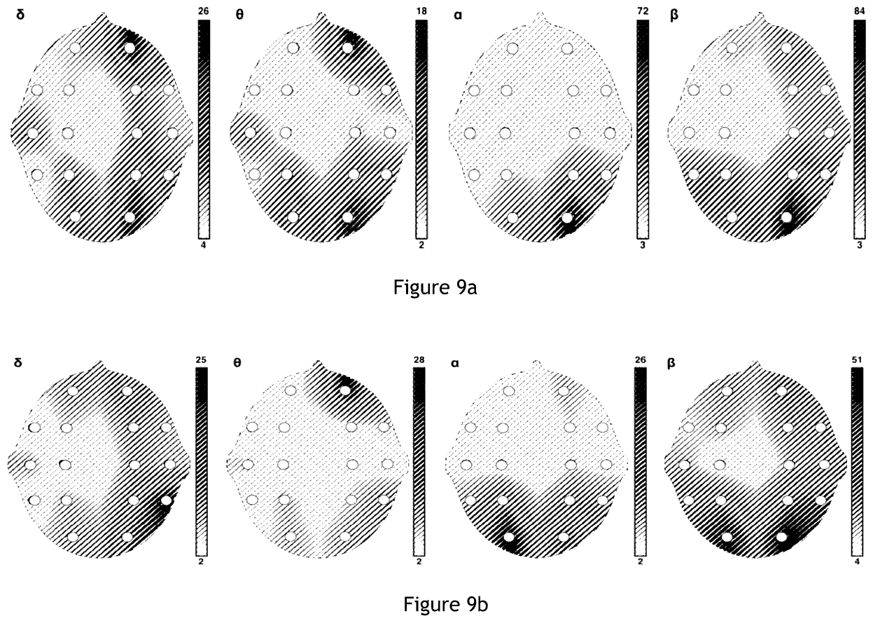

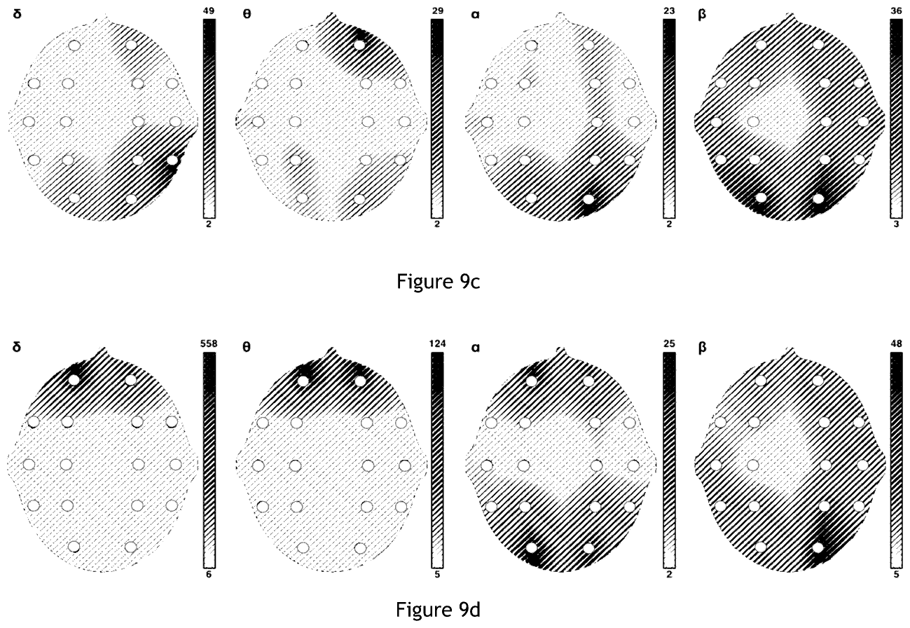

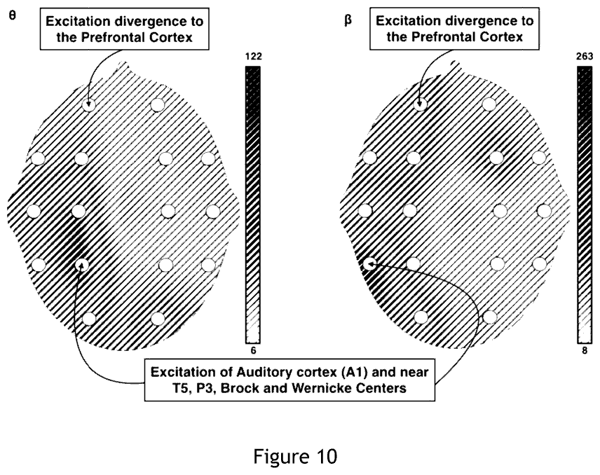

After the reading in the baseline stage, participants were invited to relax and listen to the auditory stimulus with their eyes closed. At the same time, brain neural activity was recorded (see FIG. 9A-9D).

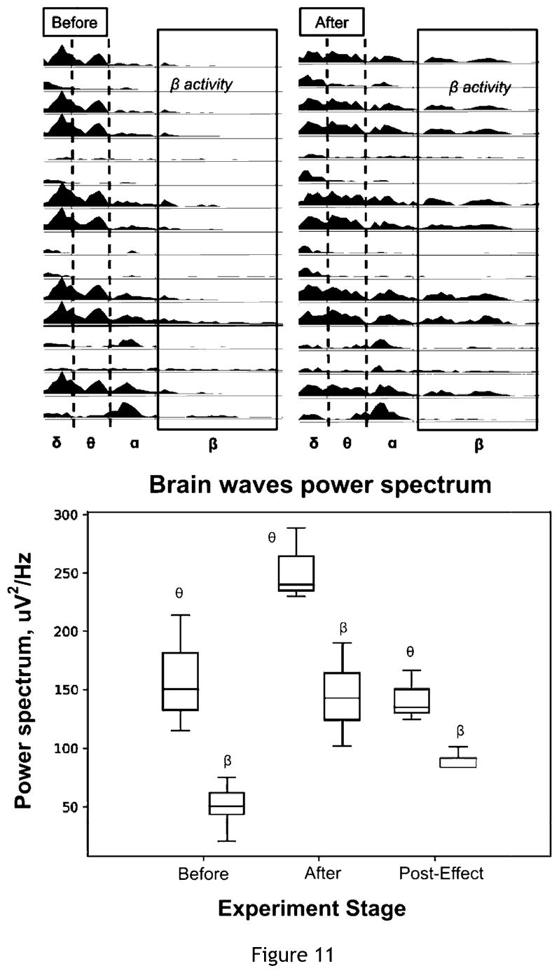

Significant and repeatable increase in spectrum power in 0 and R ranges after stimulation was observed. Changes in electrical activation localized mainly within the left temporal cortex (T7, C3, T5, P3), to smaller degree in the right temporal cortex (T4), right parietal cortex (P6), with further spread to the prefrontal cortex (Fp1, Fp2) and ventrolateral prefrontal cortex (F7, F8). Such spatial reorganization may indicate a compensation for β hypoactivity in the area of the speech centers of the brain. An averaged θ and β brain activity changes during the text reading after stimulation, related to the baseline initial reading before stimulation shown by the subtractive mapping in FIG. 11.

The mean power of 3 range increased by 3.1±0.9 fold and 0 power increased by 1.6±0.3 fold compared to the baseline state (FIG. 10).

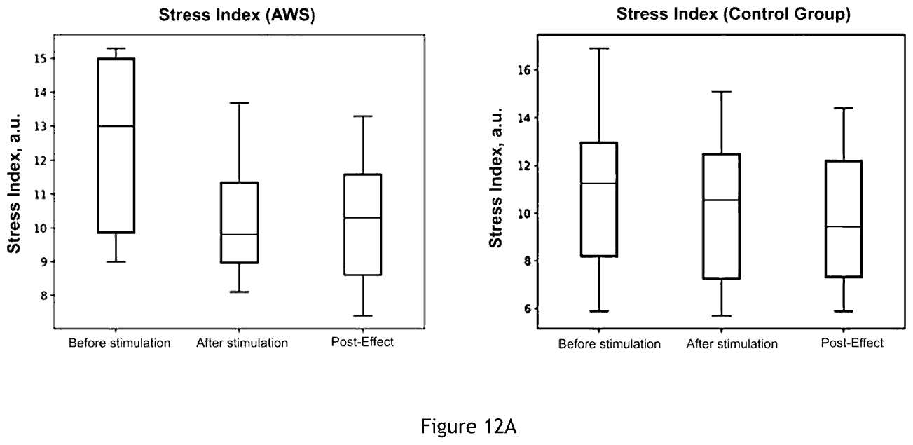

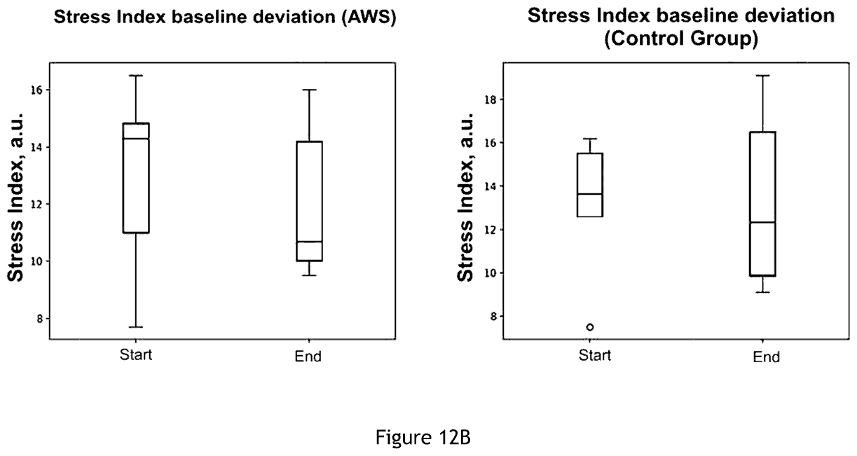

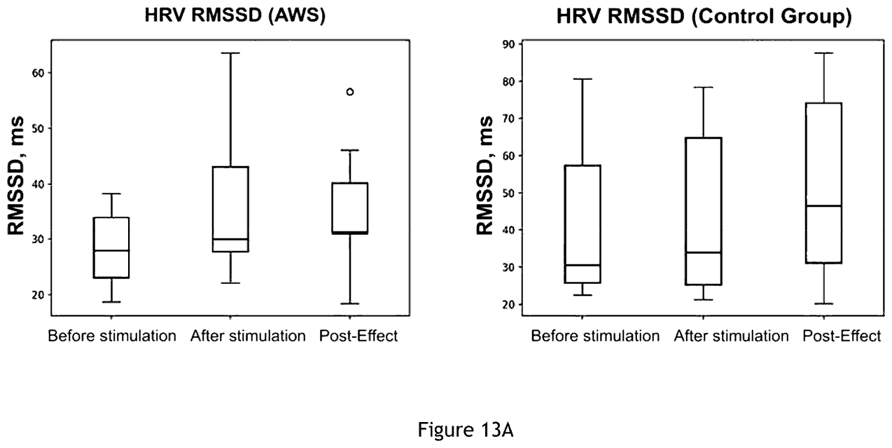

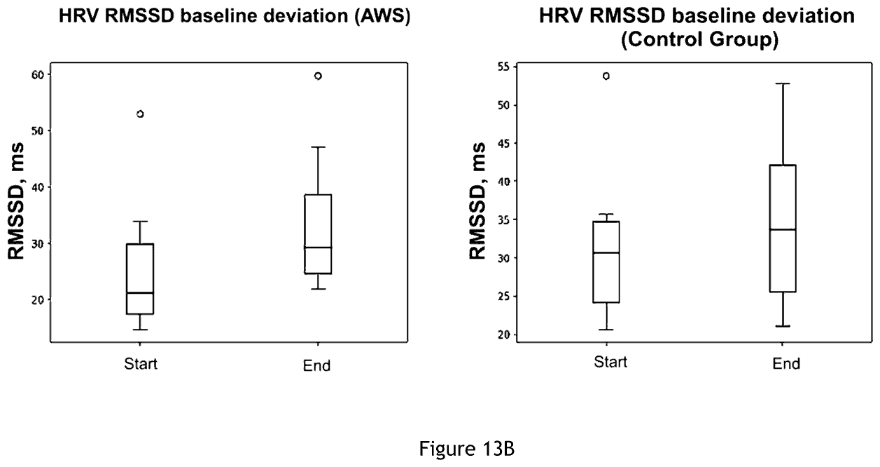

The auditory stimulation in AWS led to an increase of heart beat-to-beat variability, as evaluated by the Root Mean Square of Successive Differences (RMSSD) metric (see FIG. 12A, 12B). In an arrangement, the RMSSD may be calculated based on the difference between each heart-beat to beat (RR) interval and the previous interval. This indicator provides information relating to the heart beat dynamics, and can be used to estimate how well the body is recovering, i.e. the restoration or recovery of adaptation resources. At the same time, the Heart Rate (HR), SIdx decreases (FIG. 13A, 13B). For the current trials, observations included a balance of parasympathetic ANS and mean decrease of the SIdx by 1.4±0.3 times compared to the baseline state for the control group as well as for the AWS.

Summarizing these results, exposure to the stimulus entrains power of θ and β brain wave bands, decreases stress level and may lead to a more relaxed state and fluent speech just after stimulation. Need to be noted, that partially the positive effect of stimulation persists for a longer time from 10 and even up to 40 minutes, depending on the individual.

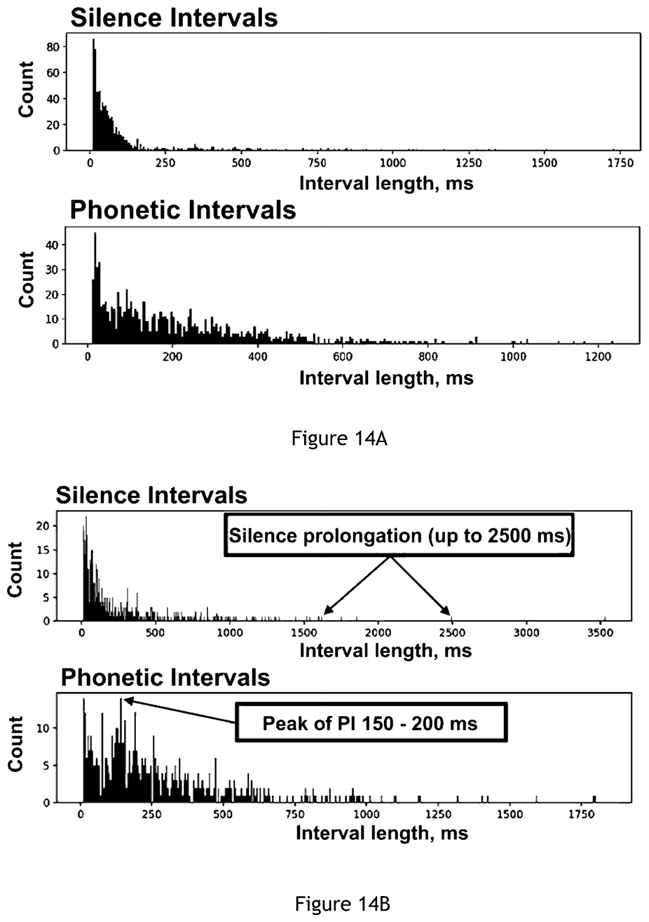

Speech analysis was performed in both control and AWS groups before and after the auditory stimulation. In order to establish the validity of speech quality metrics, we compared case and control groups using phonetic (PI) and silence interval (SI) metrics before stimulation. Clear signs of stuttering speech (FIG. 14B) were detected, compared with fluent speech (FIG. 14A) by both PI and SI metrics. Stuttering is characterized by a presence of long SIs with maximum duration up to 2,500-3,500 ms and a well-distinguished peak of PI within the 150-200 ms range.

According to the subjective assessment of researchers and participants, the speech quality in the control group did not significantly differ, while in the AWS group, immediately after stimulation, 5 out of 5 participants noted the ease of reading the text aloud. After the exposure to the stimulus, their speech lost characteristic prolongations and repetitions of phonetic constructions.

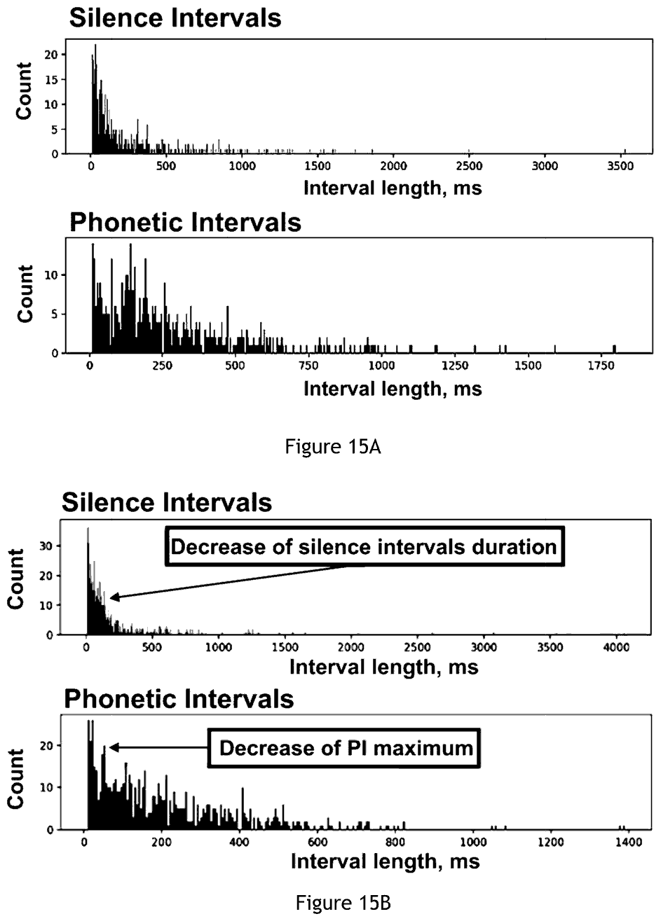

Analytically, according to the distribution of intervals (FIG. 15B) the shift of the phonetic peak to the region of up to 100 ms, as well as the absence of long pauses between phonemes compared to the before exposure speech (FIG. 15A) were demonstrated. Moreover, the distribution of intervals after the exposure is comparable to one of a fluent speaker with no stuttering (FIG. 14A).

A set of speech quality metrics was evaluated before and after the stimulus in both cohorts, showing a noticeable improvement in speech quality in AWS participants, with mild to no changes in control participants (see table below).

| Speech metrics (control group) | | | Speech metrics (AWS) | | |
|---|---|---|---|---|---|
| Metric | Before | After | Metric | Before | After |
| PI count | 930 | 941 | PI count | 542 | 712 |
| Silence count | 836 | 852 | Silence count | 522 | 707 |
| Peak PI, ms | 93 | 92 | Peak PI, ms | 139 | 51 |
| Max PI, ms | 1234 | 1144 | Max PI, ms | 1795 | 1389 |
| Max silence, ms | 1314 | 1535 | Max silence, ms | 2492 | 1650 |

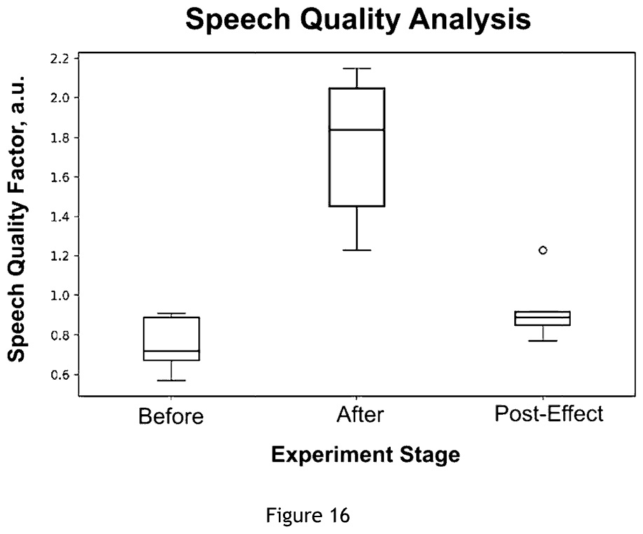

An averaged SS after stimulation increased 2.3±0.4 fold, compared to the baseline state. To monitor the stability of the stimulation effect, an additional reading test was performed after 10 minutes of rest. The results of the speech quality assessment demonstrates the post-effect of stimulation in the group of AWS. An averaged SS increased by 1.24±0.1 times, relative to the baseline (see FIG. 16). From the data above, the total number of PI increased from 470 to 560 after stimulation, the mean peak of PI decreased from 220 to 190 ms, the maximum PI decreased from 2433 to 2050 and the maximum SI decreased from 2693 to 2302 ms.

An embodiment of the present invention comprises a computer program for carrying out the above described method. Such a computer program can be provided in the form of a standard alone application, an update or add-in to an existing application or an operating system function. Methods of the present invention can be embodied in a functional library which can be called by other applications.

It will be appreciated that the above description of exemplary embodiments is not limiting and that modifications and variations to the described embodiments can be made. For example, computational tasks may be performed by more than one computing device serially or concurrently. The invention can be implemented wholly in a client computer, on a server computer or a combination of client- and server-side processing. Certain steps of methods of the present invention involve parallel computations that are apt to be implemented on processors capable of parallel computation, for example GPUs. The present invention is not to be limited save by the appended claims.

1. A method of generating an output stereo signal containing binaural beats, the method comprising:
obtaining an input audio signal having a plurality of components of different frequency;
generating a first audio signal and a second audio signal based on the input audio signal, wherein components of the first audio signal up to a frequency threshold have a frequency offset relative to corresponding components of the second audio signal;
forming the output stereo audio signal containing binaural beats from the first audio signal and the second audio signal.

2. The method of claim 1, wherein generating the first audio signal comprises generating low-frequency shifted components of the first audio signal, comprising:
generating a first shifted audio signal based on the input audio signal, by applying, in the frequency domain, a first frequency offset to the input audio signal, and
applying a low-pass filter to the first shifted audio signal.

3. The method of claim 2, wherein at least one of:
(1) the frequency range of the frequency offset is selected to avoid dissonant high-frequency artefacts,
(2) the first frequency offset is between 2 Hz and 22 Hz, and
(3) the method further comprises determining the first frequency offset on the basis of data relating to speech quality metrics of an individual.

4. (canceled)

5. (canceled)

6. The method of claim 2, wherein generating low-frequency shifted components of the first audio signal further comprises:
generating a second shifted audio signal based on the input audio signal, by applying, in the frequency domain, a second frequency offset to the input audio signal, and
applying a low-pass filter to the second shifted audio signal.

7. The method of claim 6, wherein at least one of:
(1) the second frequency offset is between 2 Hz and 22 Hz, and
(2) the method further comprises determining the second frequency offset on the basis of data relating to speech quality metrics of an individual.

8. (canceled)

9. The method of claim 6, wherein generating low-frequency shifted components of the first audio signal further comprises combining the first shifted audio signal and the second shifted audio signal together.

10. The method of claim 9, wherein one of:
(1) applying the low-pass filter to the first shifted audio signal and applying the low-pass filter to the second shifted audio signal is performed before combining the first shifted audio signal and the second shifted audio signal together, and.
(2) applying the low-pass filter to the first shifted audio signal and applying the low-pass filter to the second shifted audio signal is performed by applying a low-pass filter to the combined signal.

11. (canceled)

12. The method of claim 9, wherein generating the first audio signal comprises generating high-frequency components of the first audio signal based on the input audio signal by applying a high pass filter to the input audio signal.

13. The method of claim 12, wherein generating the first audio signal comprises combining the low-frequency shifted components and the high-frequency components together.

14. The method of claim 1, wherein at least one of:
(1) the method further comprises normalising the output stereo audio signal,
(2) one or more of the method steps are carried out in the frequency domain,
(3) one or more of the method steps are carried out in the temporal domain, and
(4) the input audio signal is a musical work.

15. (canceled)

16. (canceled)

17. A system for generating a stereo output audio signal containing binaural beats, the system comprising:
an audio signal input unit, configured to obtain an input audio signal having a plurality of components of different frequency;
a processing unit configured to generate a first audio signal and a second audio signal based on the input audio signal, wherein components of the first audio signal have a frequency offset relative to corresponding components of the second audio signal; and
a stereo output audio signal generating unit, configured to form the stereo output audio signal containing binaural beats from the first audio signal and the second audio signal.

18. The system of claim 17, wherein the processing unit is configured to generate low-frequency shifted components of the first audio signal, wherein to generate the low-frequency shifted components, the processing unit is configured to:
generate a first shifted audio signal based on the input audio signal, by applying, in the frequency domain, a first frequency offset to the input audio signal; and
apply a low-pass filter to the first shifted audio signal.

19. The system of claim 18, wherein at least one of:
(1) the first frequency offset is between 2 Hz and 22 Hz,
(2) the system further comprises a first frequency offset determination unit configured to determine the first frequency offset on the basis of data relating to speech quality metrics of an individual, and
(3) the input audio signal is a musical work.

20. (canceled)

21. The system of claim 18, wherein to generate the low-frequency shifted components, the processing unit is further configured to:
generate a second shifted audio signal based on the input audio signal, by applying, in the frequency domain, a second frequency offset to the input audio signal; and
apply a low-pass filter to the second shifted audio signal.

22. The system of claim 21, wherein at least one of:
(1) the second frequency offset is between 2 Hz and 22 Hz, and
(2) the system further comprises a second frequency offset determination unit configured to determine the second frequency offset on the basis of data relating to speech quality metrics of an individual.

23. (canceled)

24. The system of claim 21, wherein to generate the low-frequency shifted components, the processing unit is further configured to combine the first shifted audio signal and the second shifted audio signal together.

25. The system of claim 16, wherein one of:
(1) the processing unit is configured to apply the low-pass filter to the first shifted audio signal and apply the low-pass filter to the second shifted audio signal before being configured to combine the first shifted audio signal and the second shifted audio signal together, and
(2) the processing unit is configured to apply the low-pass filter to the first shifted audio signal and apply the low-pass filter to the second shifted audio signal by applying a low-pass filter to the combined signal.

26. (canceled)

27. (canceled)

28. (canceled)

29. (canceled)

30. A method of treatment for stuttering in an individual, comprising playing a stereo audio signal containing binaural beats to an individual.

31. (canceled)

32. The method of treatment of claim 30, further comprising:
determining the individual's baseline speech quality;
playing the stereo audio signal containing binaural beats to the individual;
determining the individual's speech quality after treatment.

33. (canceled)

34. (canceled)

35. (canceled)

36. (canceled)

37. (canceled)

38. A non-transitory storage medium storing a computer program comprising code means that, when executed by a computer system, perform the method of claim 1.
